(19) United States
(12) Patent Application Publication
(10) Pub. No.: US 2012/0295349 A1
(43) Pub. Date: Nov. 22, 2012

(54) MAMMAL DEDICATED CELL LINE FROM HUMAN HEPATOCELLULAR CARCINOMA CELL

(75) Inventors: TSUI-JUNG CHANG, Taoyuan County (TW); Chih-Hsien Chang, Taoyuan County (TW); Ya-Jen Chang, Taoyuan County (TW); Te-Wei Lee, Taoyuan County (TW); Tin-Yun Ho, Taoyuan County (TW); Chien-yun Hsiang, Taoyuan County (TW)

(73) Assignee: Institute of Nuclear Energy Research Atomic Energy Council, Executive Yuan, Taoyuan County (TW)

(21) Appl. No.: 13/555,495

(22) Filed: Jul. 23, 2012

Related U.S. Application Data
(62) Division of application No. 12/506,572, filed on Jul. 21, 2009, now abandoned.

(30) Foreign Application Priority Data
Feb. 20, 2009 (TW) 098105415

(51) Int. Cl. C12N 5/10

(52) U.S. Cl. 435/370

(57) ABSTRACT

A mammal dedicated cell line is provided, which is a HepG2 hepatocellular carcinoma cell line (HepG2/NF-kB/Luc/sr39tk)1_18 obtained by co-transformation with NF-kB/Luc and NF-kB/sr39tk. Firstly, a successfully transformed pNF-kB/Luc HepG2 cell is obtained. Then, a dedicated cell line sensitive to TPA and MTX is generated by experimental screening Next, a plasmid construct carrying pNF-kB/sr39tk genome is introduced into the dedicated cell line by means of Superfect protocol. Finally, a HepG2 cell line co-expressing NF-kB/Luc and NF-kB/sr39tk is screened with G418 and ZEOCIN, and transformation result is confirmed by luminescence and radio activity. The (HepG2/NF-kB/Luc/sr39tk)1_18 obtained is suitable to screen drug for treating liver cancer and examine these cells by bioluminescence imaging and nuclear medicine imaging.

### CROSS REFERENCE TO RELATED PATENT APPLICATION

THIS APPLICATION IS A DIVISIONAL OF AN application Ser. No. 12/506,572, FILED ON Jul. 21, 2009.

### BACKGROUND OF THE INVENTION

1. Field of the Invention

The present invention relates to a hepatocellular carcinoma cell HepG2 co-transformed with two different gene probes NF-κB/Luc and NF-κB/sr39tk, which is applicable in development of drugs for liver cancer and mechanisms thereof.

2. Related Art

Bioluminescence is a bio-light autonomously emitted by an organism in nature, and so named due to low heat energy generated in its light-emitting chemical reaction. Generation of bioluminescence can be directly visually observed or scientifically read and measured with an instrument, so that bioluminescence has became a commonly used tool in research of medical molecular biology at present. However, not all of light-emitting organisms can be used, and now only luciferase genes of bacteria, Photinus pyralis and Renilla reniformis have been cloned and used in gene expression and as reporters. Bacterial luciferase is a dimmer (80 KDa) from several aquatic bacteria and a land bacterium, light-emitting mechanism of which is an autonomous behavior without addition of substrate (e.g. luciferin). However, its disadvantages include unsuitability for expression in eukaryotic host, and low sensitivity and poor convenience in use caused by lux operon expression, such that it is unsuitable as reporter gene. Firefly luciferase is a monomer (61 KDa) from the firefly, which is capable of catalytically decomposing substrate luciferin. This system can effectively detect intracellular expression of reporter gene with high sensitivity, but has a disadvantage of short light emission time, and coenzyme A can be added in the reaction to slow decay of luminescence. Renilla luciferase is also a monomer (31 KDa) from Renilla reniformis, a coelenterate. When stimulated by external contact and touch, Renilla reniformis will emit light to scare or drive an aggressor away for self-protection. Its disadvantage is that some non-enzymatic luminescence will be generated in low amount, reducing analytical capability; therefore, it is generally used as a co-reporter gene in application.

With development of image medical science, currently available imaging instruments include computed tomography, ultrasonic, magnetic resonance imaging, single-photon emission tomography (SPECT) and positron emission tomography (PET). SPECT and PET allow analysis of biochemical reaction process in living organism. The operation principle is by some radioactive tracers which are compounds bearing radionuclide, and therefore SPECT and PET belong to nuclear medical imaging. Development of SPECT and PET, as well as advantage in analysis of body biochemical metabolism, contributes to growing development of radioactive drugs. Use of HSV-tk as reporter genes for an image can indirectly trace efficacy of therapeutic drugs. Currently, there have been several available radioactive drugs against HSV-tk, which can be divided into two classes: one is thymidine analogs, and another is acyclic guanosine derivatives of antiherpes simplex virus drugs. Biochemical principle of visualization of any radioactively traced drug is to negatively charge the traced drug via phosphorylation of HSV-1 TK to fail to penetrate through cell membrane, and HSV-1 tk expressing cell thus produced can achieve accumulation of traced drugs (e.g. ¹³¹I-FIAU, and ¹⁸F-FMAU), then, SPECT and PET are used for visualization and quantitative analysis; generally, reporter gene to be introduced in a cell often is a single reporter gene.

### SUMMARY OF THE INVENTION

The present invention is directed to a hepatocellular carcinoma cell HepG2 co-expressing luciferase gene (luc) and herpes simplex virus thymidine kinase gene (HSV-sr39tk), which is applicable in numerous imaging systems and NF-κB drug-screening platforms. The method mainly includes simultaneously introducing the two reporter genes into a single specific cell HepG2, such that in the HepG2 cell, luciferase gene (luc) and herpes simplex virus thymidine kinase gene (HSV-sr39tk) are co-expressed. In embodiment I of the present invention, a plasmid construct carrying pNF-κB/luc and pSV 3-neo is introduced into a hepatocellular carcinoma cell HepG2 for gene expression by the cell, next, cells successfully transformed with the genes are screened with G418, and then further screened with an optical imaging system Xenogene IVIS 100 for identifying cells more sensitive to 12-O-tetradecanoylphorbol-13-acetate (TPA) and Methotrexate (MTX) (No. L), finally, cell subculture is performed. The resulting cells No. L are subjected to a second gene transformation of a plasmid carrying pNF-κB/sr39tk and pIRESbleo3, likewise, successfully transformed cells are screened with antibiotic ZEOCIN after introduction of the plasmid. Finally, screening with antibiotics G418 and ZEOCIN is performed to identify cell lines co-expressing pNF-κB/luc and pNF-κB/sr39tk. In embodiment II of the present invention, it is demonstrated that the cell lines obtained in embodiment I can be applied in screening platform for anticancer drug, in which HepG2 NF-κB/Luc/sr39tk cells are treated with drug GCV (ganciclovir) first, and an obvious cytotoxic effect is achieved, then absorption capability for ¹³¹I-FIAU is determined, and it is found that a best absorption capability occurs in 1_{—}18 cell line, therefore, 1_{—}18 cell line is a successfully co-transformed cell line, and also has a best screening effect for anticancer drugs, and has been deposited in Food Industry Research & Development Institute with a scientific name HepG2 hepatocellular carcinoma cell line (HepG2/NF-κB/Luc/sr39tk)1_{—}18 co-transformed with NF-κB/Luc and NF-κB/sr39tk. Advantages and efficacy of the present invention are in that (1) HepG2 cell line can co-express pNF-κB/luc and pNF-κB/sr39tk, and also be influenced by NF-κB to regulate expression of reporter gene carried thereon; and (2) different imaging systems, such as optical imaging system Xenogene IVIS system, and nuclear medicine imaging systems, such as SPECT and PET, can be used in future to achieve a potential of multimodality screening of anticancer drugs to regulate and inhibit NF-κB, as screening platform of anticancer drugs to develop a new drug.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The present invention will become more fully understood from the detailed description given herein below for illustration only, and thus are not limitative of the present invention, and wherein:

FIG. 1A shows cell morphology of a HepG2 cell line;

FIG. 1B shows cell morphology of a HepG2 NF-κB/Luc cell line;

FIG. 1C shows cell morphology of a HepG2 NF-κB/Luc/sr39tk cell line;

FIG. 2A is an original luminescence pattern in a 96-well plate;

FIG. 2B is a pattern of quantified luminescence behavior by software versus cell survival;

FIG. 3 is a luminescence imaging pattern of SCID mice inoculated with HepG2 NF-κB/Luc;

FIG. 4 shows uptake effect of ¹³¹I-FIAU in HepG2 NF-κB/Luc/sr39tk cell; and

FIG. 5 shows cytotoxic effect of GCV drug on HepG2 NF-κB/Luc/sr39tk cell.

### DETAILED DESCRIPTION OF THE INVENTION

### Embodiment I

### Screening Hep G2 NF-κB/Luc Cell Line Highly Sensitive to TPA (12-O-tetradecanoylphorbol-13-acetate) and MTX (Methotrexate)

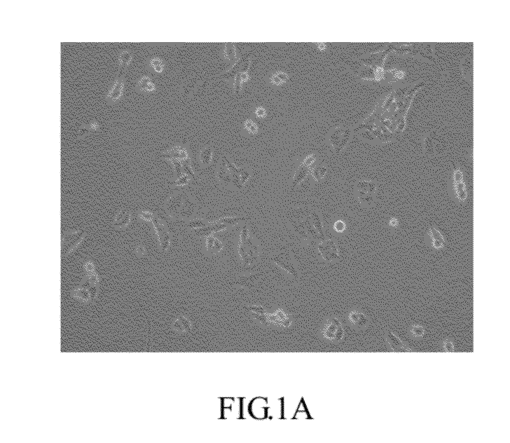

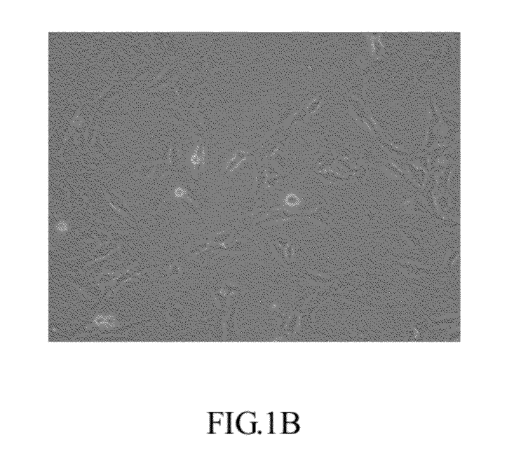

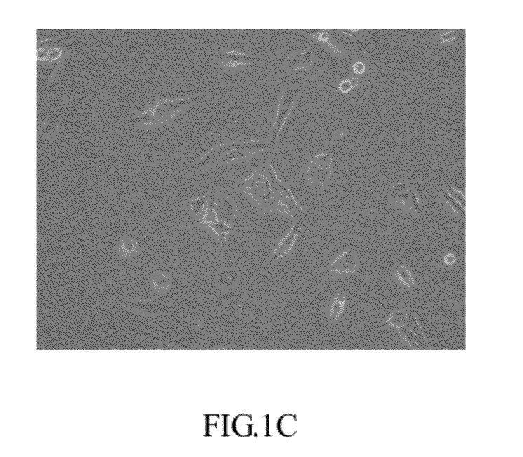

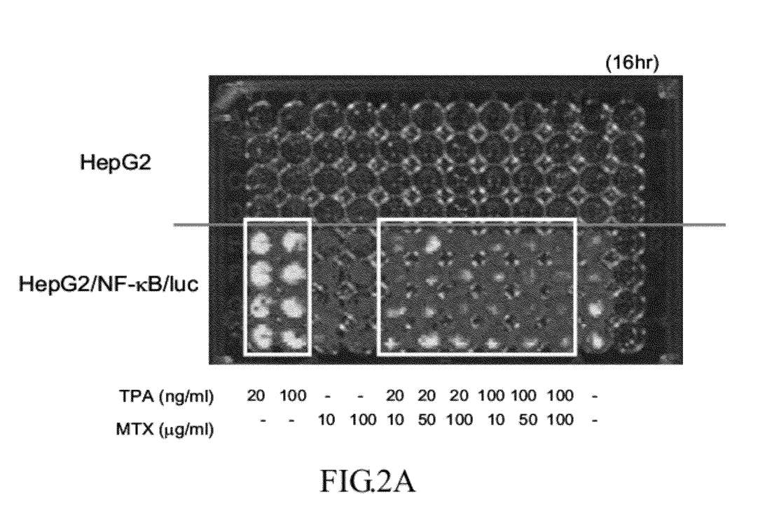

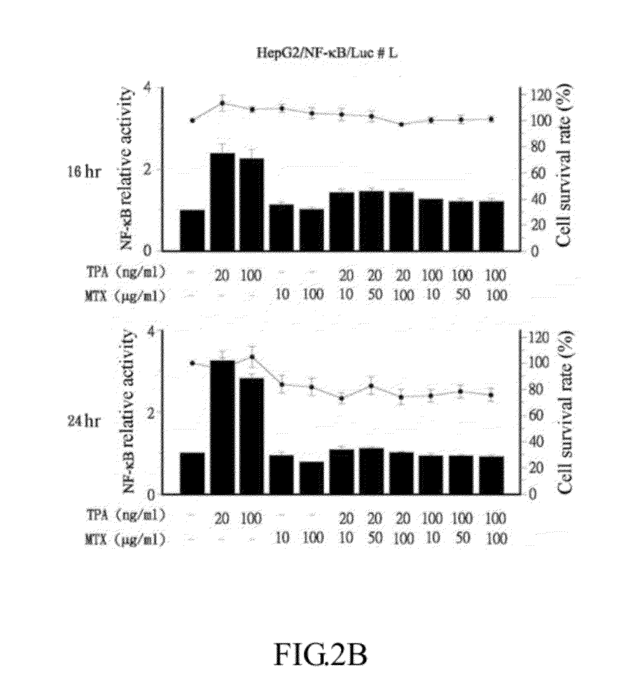

This embodiment is provided to understand inhibition of MTX on TPA-induced NF-κB activation in HepG2 NF-κB/Luc cell. First, HepG2 and HepG2 NF-κB/Luc cells (cell morphologies as shown in FIGS. 1A and 1B respectively) were inoculated in a 96-well plate at 5000 cells/200 μl/well and cultured overnight, to attach the cells onto the plate, and then treated with different concentrations of TPA (20, and 100 ng/ml), MTX (10, 50, and 100 μg/ml) and combined TPA and MTX for 16 h and 24 h. Next, 150 μg/ml of luciferin was added into the 96-well plate, which was then placed into an incubator of 37° C. and cultured for 10 min. Then luminescence behavior was detected with a luminescence/fluorescence imaging system (Xenogen IVIS 100 optical imaging system), and stronger light indicates stronger luminescence behavior (the original luminescence pattern as shown in FIG. 2A), and a quantification and analysis was preformed with a built-in software, and it was found that cell line No. L is most sensitive to TPA and MTX (the quantified luminescence pattern as shown in FIG. 2B, in which X axis represents concentrations of TPA and MTX used, Y axis on left side represents relative activity of NF-κB, and Y axis on right side represents cell survival rate).

### Building Animal Model of Luminescence Imaging with SCID Mice Inoculated with HepG2 NF-κB/Luc

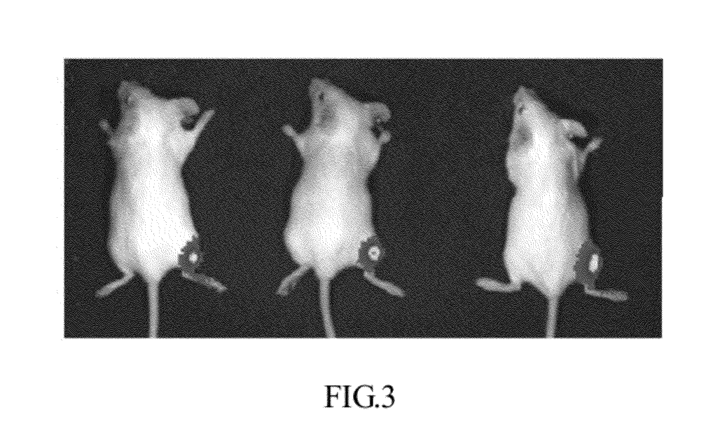

Experiment was carried out with cell line No. L, by inoculating 1.5×10⁶ cells into right leg of SCID mice by s.c., and subjecting to live animal luminescence imaging 7 days after tumor inoculation. 150 mg/kg/mouse luciferin was administrated 10 min before imaging, and then luminescence image was acquired by a luminescence/fluorescence imaging system (Xenogen IVIS 100 optical imaging system). Results show that all transplanted sites in the transplanted live animal have luminescence point, and present obvious imaging effect (luminescence imaging pattern in live animal as shown in FIG. 3).

### Transformation of NF-κB/sr39tk Gene into Cell Line No. L

5×10⁵ cells were cultured in a 25 T plate to attach the cells onto the plate before gene transformation. 0.5 μg of DNA from pNF-κB/sr39tk and pcDNA 3.1 Zeo(−) were respectively mixed with 140 μl DMEM, followed by 20 μl of SuperFect transfection reagent, and after uniformly mixed, cultured for 10 min at room temperature. Next, the DNA reactants were mixed with 1 ml of cell culture media, added into the cells, and cultured for 2 h at 37° C., then the DNA reactants were aspirated out, and repeatedly washed 4 times with 4 ml PBS, afterwards, the cells were cultured for another 48 h, and screened for successfully transformed cell line with cell culture media containing G418 and Zeocin (cell morphology of HepG2 NF-κB/Luc/sr39tk transformed cells as shown in FIG. 1C).

### Embodiment II

### Activity (131I-FIAU Uptake) Experiment of Successfully Transformed HepG2 NF-κB/Luc/sr39tk Cell

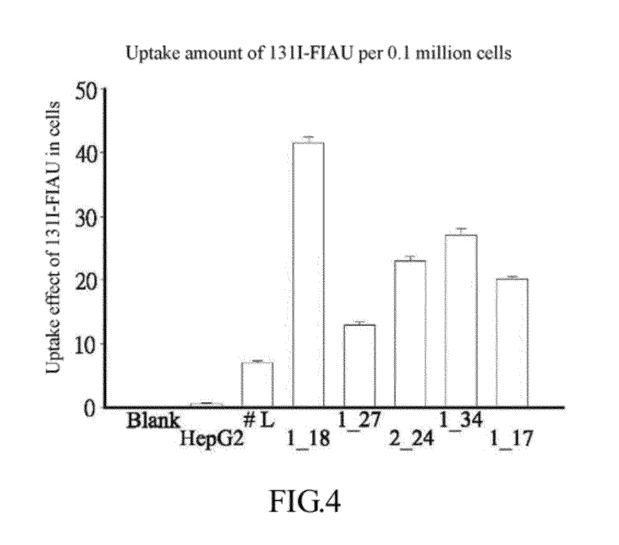

This experiment is provided to analyze uptake of 131I-FIAU drug by HepG2 NF-κB/Luc/sr39tk transformed cells. HepG2 NF-κB/Luc/sr39tk transformed cells were cultured overnight in a 6-well plate at 1×10⁶ cells/well to attach the cells onto the plate. Then, 1 ml/well of labeled ¹³¹I-FIAU was added into the cells and reacted for 2 h, and then the cells was washed with ice-cold PBS to quench the drug reaction, and detected for radioactivity. FIG. 4 is uptake effect of ¹³¹I-FIAU in HepG2 NF-κB/Luc/sr39tk cell, in which X axis represents different transformed cell line; and Y axis is uptake amount of ¹³¹I-FIAU per 0.1 million cells. Results show that HepG2 and HepG2 NF-κB/Luc cells have very low uptake efficiency for 131I-FIAU, while among the co-transformed HepG2 NF-κB/Luc/sr39tk cells, 1_{—}18 cell is most sensitive to ¹³¹I-FIAU (41.5±1.00%), followed by 1_{—}34 (27.2±1.07%), 2_{—}24 (22.9±0.68%), and 1_{—}17 (20.1±0.41%) cells, and finally 2_{—}27 (12.9±0.45%) cell.

### Cytotoxic Effect of GCV (Ganciclovir) Drug on HepG2 NF-κB/Luc/sr39tk Cell

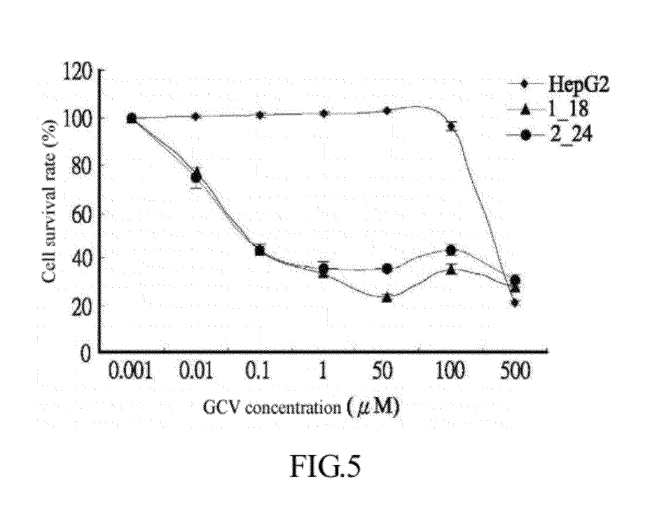

HepG2 NF-κB/luc/sr39tk was cultured at 3000 cells/well in 96 well plate format to attach the cells onto the plate, then drug-treated for 48 h with different concentrations of GCV at 0.001, 0.01, 0.1, 1, 50, 100 and 500 μM respectively, and then treated with fresh culture media containing GCV drug, and after 96 h of experiment, the culture media containing GCV drug was removed, then chromogenic reaction was carried out with WST-1 at 37° C. for 2 h, and finally analyzed by enzyme-linked immunosorbent assay (ELISA) at wavelengths of 450 nm and 620 nm respectively and plotted to obtain cell survival rate for calculation of cytotoxic effect of drug. FIG. 5 shows cytotoxic effect of GCV drug on HepG2 NF-κB/Luc/sr39tk cell, in which X axis is GCV concentration (μM); and Y axis is cell survival rate. Results show that both 1_{—}18 and 2_{—}24 exhibit obvious cytotoxic effect compared with control group (HepG2). Therefore, it is concluded from the GCV cytotoxic experiment that among the transformed cell lines, 1_{—}18 cell has maximum uptake efficiency for 131I-FIAU, thus determined to be successfully co-transformed cell line, and biologically deposited with scientific name HepG2 hepatocellular carcinoma cell line (HepG2/NF-κB/Luc/sr39tk)1_{—}18 co-transformed with NF-κB/Luc and NF-κB/sr39tk, which has evidenced survival.

What is claimed is:

1. A mammal dedicated cell line, being a HepG2 hepatocellular carcinoma cell line (HepG2/NF-kB/Luc/sr39tk)1_{—}18 obtained by co-transformation with NF-kB/Luc and NF-kB/sr39tk, and deposited in Food Industry Research & Development Institute under Accession No. BCRC960385 on Jan. 13, 2009, obtained from human hepatocellular carcinoma cell (HepG2) by co-transformation.
